(19) United States
(12) Patent Application Publication
(10) Pub. No.: US 2006/0281101 A1
(43) Pub. Date: Dec. 14, 2006

(54) Biobriefcase

(75) Inventors: John M. Dzenitis, Danville, CA (US); William J. Benett, Livermore, CA (US); Raymond P. Mariella JR., Danville, CA (US); Steven R. Visuri, Waukesha, WI (US); Kodumudi S. Venkateswaran, Livermore, CA (US)

Correspondence Address: Eddie E. Scott;Assistant Laboratory Counsel, Lawrence Livermore National Laboratory, P.O. Box 880, L-703, Livermore, CA, 94551, US

(73) Assignee: The Regents of the University of California

(21) Appl. No.: 11/154,975

(22) Filed: Jun. 15, 2005

Related U.S. Application Data
(60) Provisional application No. 60/654,634, filed on Feb. 17, 2005.

(51) Int. Cl. C12Q 1/68; G01N 33/53; C12M 1/34

(52) U.S. Cl. 435/6; 435/7.1; 435/287.2; 435/7.5

(57) ABSTRACT

A bio-briefcase system for analyzing a sample for the presence of biological agents. The bio-briefcase system comprises a housing, an immunoassay section operatively connected to the housing, and/or a nucleic acid assay section operatively connected to the housing.

### CROSS-REFERENCE TO RELATED APPLICATIONS

This application claims the benefit of U.S. Provisional Patent Application No. 60/654,634 titled “Biobriefcase” filed Feb. 17, 2005 by John M. Dzenitis, William Benett, Raymond Mariella, Steven R. Visuri, and Kodumudi S. Venkateswaran. U.S. Provisional Patent Application No. 60/654,634 titled “Biobriefcase” filed Feb. 17, 2005 is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

Field of Endeavor

The present invention relates to biological pathogens and more particularly to a bio-briefcase system for detecting biological pathogens.

State of Technology

There exists a critical need to develop distributed biothreat agent sensor networks that can operate in civilian applications. To operate in “Detect to Protect/Warn” type detection architectures, these platforms need to have several key properties. They need to be capable of detecting pathogens within a 1-2 hour time window, allowing for enough time to respond to an event. They need to be extremely low cost to maintain, since continuous monitoring is essential for many applications. These platforms need to have sufficient sensitivity to cover a broad geographical area (limiting the necessary number of sensors) and have sufficient selectivity to virtually eliminate false positives. Currently available bio-weapons detection systems are designed primarily for military use on the battlefield. These systems are often expensive to deploy and ultimately unsuited for civilian protection.

In an article titled, “U.S. Is Deploying a Monitor System for Germ Attacks,” by Judith Miller in The New York Times on Jan. 22, 2003, it was reported, “To help protect against the threat of bioterrorism, the Bush administration on Wednesday will start deploying a national system of environmental monitors that is intended to tell within 24 hours whether anthrax, smallpox and other deadly germs have been released into the air, senior administration officials said today. The system uses advanced data analysis that officials said had been quietly adapted since the September 11 attacks and tested over the past nine months. It will adapt many of the Environmental Protection Agency's 3,000 air quality monitoring stations throughout the country to register unusual quantities of a wide range of pathogens that cause diseases that incapacitate and kill. . . . The new environmental surveillance system uses monitoring technology and methods developed in part by the Department of Energy's national laboratories. Samples of DNA are analyzed using polymerase chain reaction techniques, which examine the genetic signatures of the organisms in a sample, and make rapid and accurate evaluations of that organism. . . . Officials who helped develop the system said that tests performed at Dugway Proving Ground in Utah and national laboratories showed that the system would almost certainly detect the deliberate release of several of the most dangerous pathogens. ‘Obviously, the larger the release, the greater the probability that the agent will be detected,’ an official said. ‘But given the coverage provided by the E.P.A. system, even a small release, depending on which way the wind was blowing and other meteorological conditions, is likely to be picked up.’”

In an article titled, “Biodetectors Evolving, Monitoring U.S. Cities,” by Sally Cole in the May 2003 issue of Homeland Security Solutions, it was reported, “The anthrax letter attacks of 2001, and subsequent deaths of five people, brought home the reality of bioterrorism to Americans and provided a wake-up call for the U.S. government about the need for a method to detect and mitigate the impact of any such future attacks. Long before the anthrax letter attacks, scientists at two of the U.S. Department of Energy's national laboratories, Lawrence Livermore National Laboratory (LLNL) and Los Alamos National Laboratory (LANL), were busy pioneering a “biodetector” akin to a smoke detector to rapidly detect the criminal use of biological agents. This technology is now expected to play a large role in the U.S. government's recently unveiled homeland security counter-terrorism initiative, Bio-Watch, which is designed to detect airborne bioterrorist attacks on major U.S. cities within hours. Announced back in January, Bio-Watch is a multi-faceted, multi-agency program that involves the U.S. Department of Energy, the Environmental Protection Agency (EPA), and the U.S. Department of Health and Human Services' Centers for Disease Control and Prevention (CDC). Many of the EPA's 3,000 air-quality monitoring stations throughout the country are being adapted with biodetectors to register unusual quantities of a wide range of pathogens that cause diseases that incapacitate and kill, according to the EPA. The nationwide network of environmental monitors and biodetectors, which reportedly will eventually monitor more than 120 U.S. cities, is expected to detect and report a biological attack within 24 hours. Citing security reasons, the EPA declined to disclose further details about the program at this time. . . . The Autonomous Pathogen Detection System (APDS) is file-cabinet-sized machine that sucks in air, runs tests, and reports the results itself. APDS integrates a flow cytometer and real-time PCR detector with sample collection, sample preparation, and fluidics to provide a compact, autonomously operating instrument capable of simultaneously detecting multiple pathogens and/or toxins. The system is designed for fixed locations, says Langlois, where it continuously monitors air samples and automatically reports the presence of specific biological agents. APDS is targeted for domestic applications in which the public is at high risk of exposure to covert releases of bioagents—subway systems, transportation terminals, large office complexes, and convention centers. . . . APDS provides the ability to measure up to 100 different agents and controls in a single sample,’ Langlois says. ‘It's being used in public buildings right now.’ The latest evolution of the biodetector, APDS-II, uses bead-capture immunoassays and a compact flow cytometer for the simultaneous identification of multiple biological stimulants. Laboratory tests have demonstrated the fully autonomous operation of APDS-II for as long as 24 hours.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a bio-briefcase system for analyzing a sample for the presence of biological agents, the bio-briefcase system comprises housing and various assay sections, potentially including an immunoassay section operatively connected to the housing and/or a nucleic acid assay section operatively connected to the housing. An input opening to the bio-briefcase system can utilize the port for injection of a fluid-based sample or the input port can be utilized to draw a liquid or gas from an aerosol-to-liquid collector. The sample can be directed to the immunoassay section and to the nucleic acid assay section. The sample may be analyzed for nucleic acids (DNA or RNA) characteristic of pathogens, or analyzed for characteristic proteins via affinity assays (antigen/antibody). The original sample may be split to be analyzed simultaneously by both methods, or analyzed by one method first with the second method acting as a confirmation of prior results.

Biological weapons have been used in warfare for centuries, but the threat to the civilian population has become particularly acute within the last decade. Incidents such as the anthrax letter attacks and the Sarin gas attack in the Tokyo subway have made clear the need to be prepared for future chem/bio-warfare releases. Preparation should include efforts from prevention through detection and treatment. The bio-briefcase system of the present invention comprises a briefcase-sized case 1 enabling the bio-briefcase system to be easily hand-carried onto a plane or loaded into a vehicle for rapid deployment when and where needed. The small sizes also enable less intrusive installation in air-handling ducts, public passages, or covert monitoring applications. The components immunoassay and nucleic acid assay of the bio-briefcase system are operatively connected to and carried by the case. The bio-briefcase system provides a briefcase-sized instrument capable of detecting the full spectrum of biowarfare agents. The bio-briefcase system is capable of processing liquid samples or suspensions of solids (e.g., cells) in a liquid. The organisms that can include proteins (e.g., toxins), bacteria, or viruses. The bio-briefcase system can be used to limit or prevent civilian exposure to biological pathogens, and initiate early treatment for those exposed; thereby decreasing mortality and morbidity from bioterrorism events.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is an illustration of one embodiment of a bio-briefcase system for performing analysis of a sample for the presence of biological agents.

FIG. 2 is a schematic illustration of one embodiment of a bio-briefcase system for performing analysis of a sample for the presence of biological agents.

FIG. 3 is a schematic illustration of the immunoassay train of one embodiment of a bio-briefcase system for performing analysis of a sample for the presence of biological agents.

FIG. 4 is a schematic illustration of the nucleic acid assay train of one embodiment of a bio-briefcase system for performing analysis of a sample for the presence of biological agents.

### DETAILED DESCRIPTION OF THE INVENTION

Referring now to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

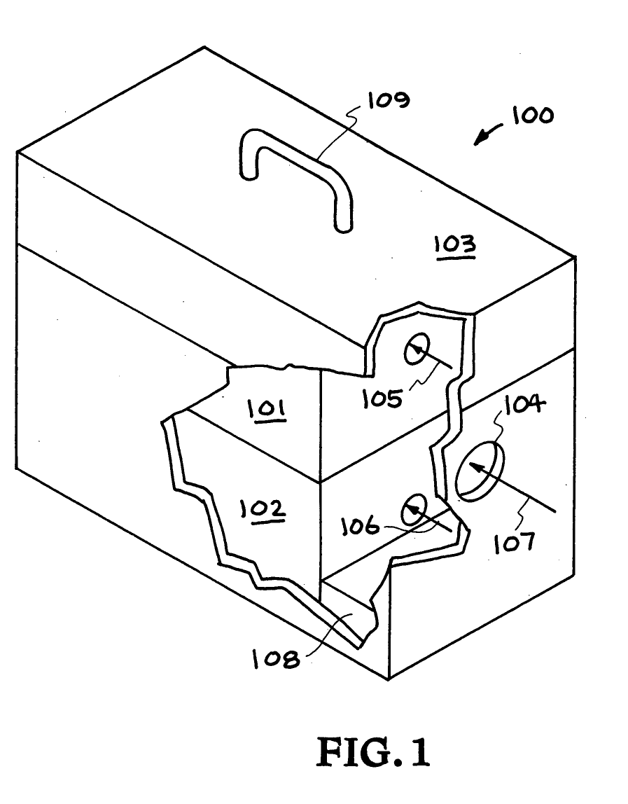

Referring now to the drawings, and in particular to FIG. 1, an illustration of one embodiment of a bio-briefcase system for performing analysis of a sample for the presence of biological agents is shown. The bio-briefcase system is designated generally by the reference numeral 100. The bio-briefcase system 100 includes the following structural components: immunoassay section 101, nucleic acid assay section 102, case 103 that forms a housing, input opening 104, input 105 to immunoassay section, input 106 to nucleic acid assay section, power unit 108, and handle 109.

The bio-briefcase system 100 comprises a briefcase-sized case 103 having a hard polyethylene shell. The case 103 provides structural support to the immunoassay section 101 and nucleic acid assay section 102. The case 103 positions and maintains the power unit 108, the input opening 104, the input 105 to the immunoassay section, and the input 106 to the nucleic acid assay section in the proper position to operate the bio-briefcase system 100 during all phases of operation and transport. The case 103 is sufficiently small (size 2′×1′×1′ or smaller), enabling the bio-briefcase system 100 to be easily hand-carried onto a plane or loaded into a vehicle for rapid deployment when and where needed. The small sizes also enable less intrusive installation in air-handling ducts, public passages, or covert monitoring applications. The case 103 and the bio-briefcase system 103 provides a broad-spectrum bioagent detector that is briefcase-sized and features dramatically reduced reagent consumption, improved sensitivity and rapid response time. Sample preparation and analysis is carried out on microfluidic, chip-based modules. The briefcase-sized system uses DNA amplification to identify bacteria and viruses using polymerase chain reaction (PCR) and uses immunoassays to identify bacteria, viruses, toxins; and protein signatures to identify toxins. Serving as an environmental monitor, it functions autonomously to collect and detect samples in a stealthy and easily deployed manner. The BioBriefcase is also capable of being manned by a minimally trained user to function as a portable laboratory, providing quick turn-around between sample analysis and responsive action.

The two structural components immunoassay 101 and nucleic acid assay 102 of the bio-briefcase system 100 are operatively connect to and carried by the case that forms a housing 103. The bio-briefcase system 100 provides a briefcase-sized instrument (BioBriefcase) capable of detecting the full spectrum of biowarfare agents (bacteria, viruses and toxins). The bio-briefcase system 100 is capable of processing liquid samples or suspensions of solids (e.g., cells) in a liquid. The organisms that can be detected include proteins (e.g., toxins), bacteria, or viruses.

The input opening 104 to the bio-briefcase system 100 can utilize the port 104 for injection of a fluid-based sample or the input port 104 can be utilized to draw a liquid or gas 107 from an aerosol-to-liquid collector. The sample is directed to input 105 and to the immunoassay section 101, to input 106 and to the nucleic acid assay section 102. Following input of the sample several steps are taken to perform analysis of the sample prior to detection. The sample may be analyzed for nucleic acids (DNA or RNA) characteristic of pathogens, or analyzed for characteristic proteins via affinity assays (antigen/antibody). The original sample may be split to be analyzed simultaneously by both methods, or analyzed by one method first with the other method acting as a confirmation of prior results.

The sample pathway for nucleic acid analysis consists of some or all of the following steps: lysis, concentration, purification, amplification, detection. If cells or spores are expected, mechanical or chemical lysis may be performed to access the nucleic acids more readily. Mechanical lysis may consist of applying acoustic energy to the sample with or without the aid of cavitation enhancers (bubbles, beads, impurities) or beads for collisional cavitation. Chemical lysis can be performed with a variety of agents that will cause cells to lyse or spores to begin to germinate for easier lysis.

If a large or “dirty” sample is expected the sample may be concentrated and purified. Cells or spores may be concentrated by filtering the liquid and discarding excess. Nucleic acids may be “exposed” by using choatropic agents. Exposed nucleic acids can be concentrated by capturing them out of solution onto a solid substrate. The capture substrate can be any material with an affinity (hydrogen bonding, electrostatic attraction, etc.) for nucleic acids such as glass or silicon dioxide. The excess solution which may contain contaminants that interfere with the assays can then be washed away while the nucleic acids are retained. Subsequently the nucleic acids can be released by altering the properties of the buffer (elution) solution (e.g., pH or ionic strength change).

If low concentrations of sample are to be detected an amplification step is useful. PCR is one established method of amplifying the sample and can be easily accommodated into a microfluidic flow-though instrument. PCR products can be detected by several methods such as direct labeling with fluorescent dyes (intercalating or sequence-labeled), indirect methods (Taqman), or non-optical means (e.g., electrochemical). Multiplexed samples require additional strategies to enable individual detection of multiple targets. This may be accomplished with unique tags for each nucleic acid signature. Multiplex optical detection may be accomplished with spectrally resolved dyes, upconverting phosphors, quantum nanocrystals (quantum dots), or ratios of dyes and dots encapsulated into a solid tag. An alternate approach is to physically separate the species and use location as the discriminator. Separation may be accomplished by capillary electrophoresis which sorts by mass and charge differences. Target probes can be bound to electrophoretic tags (e.g., ACLARA's eTags). The tags can be released by nuclease activity when the probe binds to the target sequence. The free tags can then be separated by CE and detected as an alternative to direct detection of amplified product. CE separation can be detected by laser-induced fluorescence, absorbance, or electrochemically. DNA arrays are another approach to physical separation of targets.

The sample pathway for immunologic analysis consists of some or all of the following steps: lysis, concentration, purification, hybridization, detection. Lysis would be performed as described above. Sample concentration and purification may occur by extracting proteins or other species out of solution. This may be performed by binding to a solid substrate that is fixed (treated surface) or in solution (beads). The waste can then be discarded while retaining the desired target sample. Other means exist for concentration of protein antigens. For the detection, specific antibody (immunological) stains can be used to mark the proteins of interest. An alternate method is to couple the antibodies for the proteins to electrophoretic markers (e.g., ACLARA's eTags). A second antibody is bound to a molecular scissors that consists of photoactivated porphyrins. When a target antigen is present, the eTag labeled antibody and the porphyrin antibody both bind to the antigen. By exposing the porphyrin to light, singlet oxygen is produced and the eTag is subsequently released. The eTags can then be separated and detected using CE.

The system 100 addresses current needs and improves upon previous instruments in many ways. The system 100 decreases size and reagent requirements and improves automation and function. There is great demand for small scale instrumentation for biological agent detection, both in the medical and national security communities. A point-of-care instrument that can simultaneously detect multiple organisms is highly useful in hospitals, doctor's offices, field medicine settings, and similar uses. Likewise, a portable detector is highly useful to personnel responding to biowarfare incidents. Also, an instrument that continuously monitors environmental samples is highly useful for national security. While many instruments currently exist to address portions of these applications, there are drawbacks inherent with them all. There is no known instrument that performs sensitive, multiplex detection in a portable cost-efficient platform.

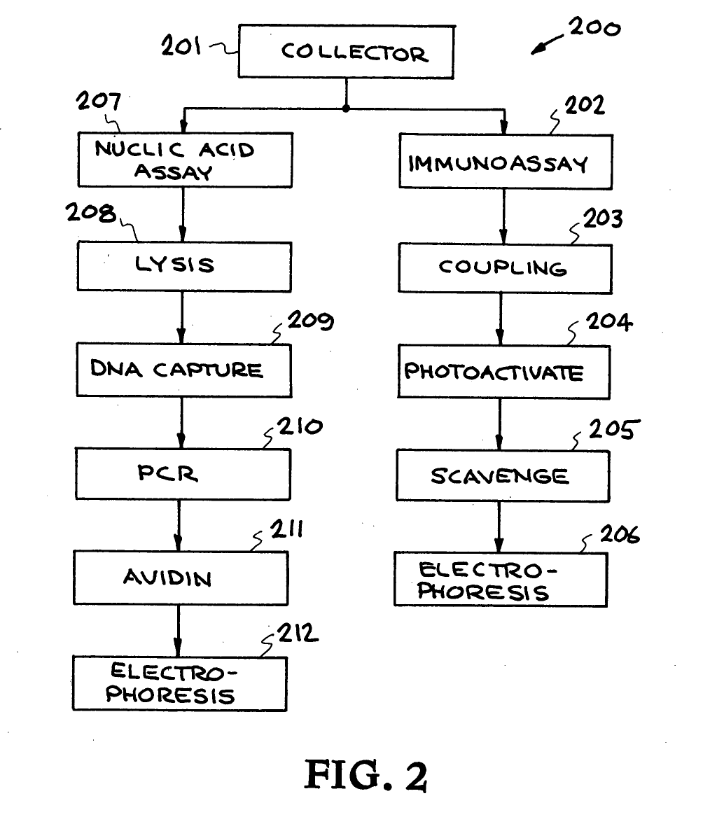

Referring now to FIG. 2, a schematic illustration of one embodiment of a bio-briefcase system for performing analysis of a sample for the presence of biological agents is shown. The bio-briefcase system is designated generally by the reference numeral 200. The system 200 may be run completely autonomously for continued monitoring of the environment (air or water) or can be operated on-demand by a user (injected samples). The system 200 is capable of processing liquid samples or suspensions of solids (e.g., cells) in a liquid. The organisms that can be detected include proteins (e.g., toxins), bacteria, or viruses.

The bio-briefcase system 200 includes the two analysis trains: immunoassay 202 and nucleic acid assay 207. The bio-briefcase system 200 uses the two separate and completely orthogonal analysis trains—immunoassay 202 and nucleic acid assay 207—to provide two analysis methods each for the detection of bacteria, viruses, and toxins. In the case of toxins and PCR, the agent is not detected directly but residual DNA from the organism of origin can be detected. Both analysis modes will employ capillary electrophoresis for detection.

The bio-briefcase system 200 includes the following individual structural components: collector 201, immunoassay 202, coupling 203, photoactivate 204, scavenge 205, electro-phoresis 206, nucleic acid assay 207, lysis 208, DNA capture 209, PCR 210, avidin 211, and electro-phoresis 212. The system 200 addresses current needs and improves upon previous instruments in many ways. The system 200 decreases size and reagent requirements and improves automation and function. There is great demand for small scale instrumentation for biological agent detection, both in the medical and national security communities. A point-of-care instrument that can simultaneously detect multiple organisms is highly useful in hospitals, doctor's offices, field medicine settings, and similar uses. Likewise, a portable detector is highly useful to personnel responding to biowarfare incidents. Also, an instrument that continuously monitors environmental samples is highly useful for national security. While many instruments currently exist to address portions of these applications, there are drawbacks inherent with them all. There is no known instrument that performs sensitive, multiplex detection in a portable cost-efficient platform.

The bio-briefcase system 200 utilizes emerging technology of fluorescent electrophoretic tags for multiplex detection. These tags can be coupled to antibodies or nucleic acid probes, thus enabling both immuno- and PCR/nucleic acid-assays. Following a recognition event, the single color electrophoretic tags are separated and detected using capillary electrophoresis. Preliminary data demonstrates that it is easily possible to multiplex these markers and achieve immunoassay limits of detection that are equivalent to or better than standard ELISA assays. By having dual detection strategies, false positives will be greatly reduced.

The bio-briefcase system 200 uses electrophoretic mobility tag (eTag) technology that currently affords the ability to run 40 simultaneous assays with a single laser excitation source for detection. By adding a second color excitation source, the multiplicity is doubled. This allows for broad coverage of many possible agents of bioterrorism.

Background of eTag-“eTag” is short for electrophoretic tag, part of a detection process invented and commercialized by ACLARA BioSciences. The main purpose is to multiplex assays in a single vessel with all materials in solution. The current embodiment of eTag reporters is a set of fluorescein-derived compounds with distinct electrophoretic mobilities that allow a high degree of multiplexing within a single solution. A key feature of the eTag process is that it disconnects the molecular biology recognition event (antibody binding or PCR amplification) from the detection event (capillary electrophoresis). Consequently, the eTag reporters can be optimized to the detection needs independently of the reagents optimized for the bioassay. The biobriefcase uses eTag reporters to serve a role similar to Luminex beads on the Autonomous Pathogen Detection System. Advantages of the eTag reporters include the following: no solid phase (bead) is involved, which greatly simplifies sample handling and improves reliability; very little liquid needs to be transferred, which minimizes the volumes of reagents required; multiplex detection comes from a single color, which simplifies the detection system; and high quantum efficiency dyes can be used as eTags, increasing detection sensitivity, and reducing excitation requirements.

In an immunoassay, an antibody labeled with an eTag (e.g., fluorescein derivative) and a secondary antibody labeled with molecular scissors (e.g., porphyrin) are used. These antibodies may or may not be otherwise identical. A solution containing, for example, nanomolar concentrations of antibodies would be mixed with a solution with as little as picomolar concentrations of target. When both antibodies find a target and form a sandwich similar to ELISA, the eTag and its linker are brought in close proximity to the molecular scissors. The solution is treated to activate the molecular scissors and free the eTag. In this case, the porphyrin (scissors) is photoactivated with 635 nm light, producing singlet oxygen that cleaves the eTag-antibody linker. The solution is subsequently treated (proprietary method) to immobilize the eTags that were not released, and direct electrokinetic injection of the freed eTags (negatively charged) into capillary electrophoresis is performed. Since many different eTags (with the same “color”) can be distinguished in one analysis, many distinct immunoassays can be performed simultaneously.

The bio-briefcase system 200 is illustrated schematically in FIG. 2 and consists of the two analysis trains immunoassay 202 and nucleic acid assay 207. The front-end aerosol collector and back-end CE separation/detection is common to both trains. In between collection and detection are the sample preparation modules that perform hybridization, filtering, concentration, and amplification.

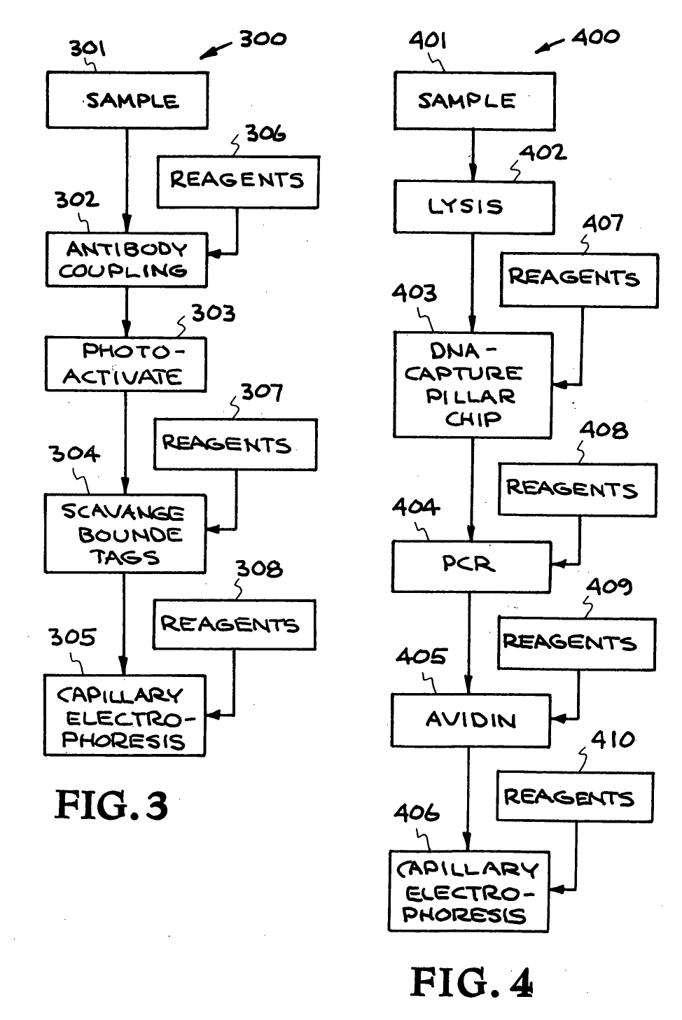

Referring now to FIG. 3, a schematic illustration of the immunoassay train of one embodiment of a bio-briefcase system for performing analysis of a sample for the presence of biological agents is shown. The immunoassay train is designated generally by the reference numeral 300. The schematic illustration of the immunoassay train 300 includes the following components: sample 301, antibody coupling 302, photoactivate 303, scavenge bound eTags 304, capillary electro-phoresis 305, reagents 306, reagents 307, and reagents 308.

The structural components of the immunoassay train 300 having been described and illustrated in FIG. 3, the construction and operation of the immunoassay train 300 will now be described. The construction and operation of the immunoassay train 300 includes various mixing, moving, and sample preparation operations. The mixing and moving the sample 301 is performed by microfluidics. A heated incubation chamber may improve the speed or total number of antibodies bound to an antigen. Light exposure to activate the photosensitive porphyrin can be supplied by red LED's.

The sample 301 obtained from the aerosol collector or other system is mixed with antibody reagent 306 (bound to eTags). This may possibly be accomplished by providing a heated incubation chamber. The mixture is moved to the photoactivate section 303 where it is photoactivated by exposing the mixture to light (to cleave eTags bound to antigen). The mixture is next mixed with reagents 307, such as Avidin, to scavenge eTags that remain bound to antibodies. The mixture is next moved to the capillary electro-phoresis section 305. The capillary electro-phoresis is common to both immunoassay and nucleic acid assay. The capillary electro-phoresis as used in capillary electro-phoresis 305 is described below.

Capillary Electrophoresis—Capillary electrophoresis (CE) was chosen as the platform for detection because it has many advantages. Some of the advantages are: proven sensitive multiplex detection method, amenable to flow through, automated assay performance (no need to exchange disposable after every assay), scalable (chip-based CE demonstrated by many groups and companies), minimal reagent requirements (lower cost), applicable to nucleic acid- and immuno-assays (coverage for all classes of bioagents, plus enables dual/confirming detection mode), and solution-based chemistry, no bead handling issues.

Referring now to FIG. 4, a schematic illustration of the nucleic acid assay train of one embodiment of a bio-briefcase system for performing analysis of a sample for the presence of biological agents is shown. The nucleic acid assay train is designated generally by the reference numeral 400. The schematic illustration of the nucleic acid assay train 400 includes the following components: sample 401, lysis 402, DNA-capture pillar chip 403, PCR 404, avidin 405, capillary electro-phoresis 406, reagents 407, reagents 408, reagents 409, and reagents 410.

The structural components of the nucleic acid assay train 400 having been described and illustrated in FIG. 4, the construction and operation of the nucleic acid assay train 400 will now be described. The construction and operation of the nucleic acid assay train 400 includes various mixing, moving, and sample preparation operations. The mixing and moving the sample 401 is performed by microfluidics.

Sample preparation for the nucleic acid assay train involves several steps. The sample 401 is moved to the lysis section 402. In this section, lysing the sample 401 is completed. Most biological organisms such as viruses and bacteria can be easily heated to release genomic DNA. However, bacterial spores such as Bacillus anthracis are resistant to both heat and chemical disruption. Previous studies have shown that spore lysis can greatly increase the availability of DNA and therefore dramatically improve the sensitivity of PCR-based assays. A flow-through lysis module can be used or an alternate mechanical method of spore lysis based on acoustics can be used. After lysis, the sample 401 is filtered to filter the lysate to remove large particles and concentrated to concentrate the sample (to improve sensitivity).

The sample 401 is next moved to the DNA-capture pillar chip section 403. PCR is an amplification technique that yields extremely sensitive detection, down to single copy if performed carefully and efficiently. It is advantageous to perform PCR on very small sample volumes to conserve PCR reagents. These two goals, sensitivity and small sample size, are often at odds, as limited sampling increases the chance that target DNA will not be present in an aliquot. Further, environmental contaminates may be present that inhibit PCR and decrease sensitivity. The BASIS project found that locations such as subway terminals contain high levels of aerosolized metals that inhibit PCR. The DNA-capture pillar chip section 403 includes a DNA capture chip that concentrates and filters DNA from large sample volumes to address these issues. The DNA-capture pillar chip section 403 is a silicon “pillar chip” that employs solid-phase reversible immobilization of DNA, similar to the labor intensive extraction techniques used by molecular biologists for over a decade. The DNA capture chip was developed for automated flow-through processing. The chip consists of densely packed microfabricated silicon pillars, designed to increase the surface area (reaction area). By properly controlling the buffer chemistry, DNA can be “salted” out of solution and preferentially bind to the pillars. A buffer exchange causes the DNA to elute out of the chip. The chip can process samples at greater than 1 ml/min and elute sample into <10 μl aliquots.

The DNA-capture pillar chip section 403 has been used to demonstrate >12-fold concentration of Erwinia herbicola bacteria by condensing a 1 ml sample into 20 μl. Similarly, the DNA-capture pillar chip section 403 has been used to demonstrate approximately 1000-fold concentration of Fracisella tularensis by processing 75 ml of sample.

The sample 401 is next moved to the PCR section 404. The sample 401 is filtered to remove PCR inhibitors. The PCR section 404 is used to perform PCR amplification. The PCR amplification can be flow-through PCR and the PCR section 404 can be a PCR thermal cycler. The PCR section 404 can be a silicon-based thermal chamber is small and rapid. This technology has been adapted to a flow-through format and has been demonstrated to amplify with >90% efficiency, detect down to single copy concentration, and compare favorably to performing PCR in the SmartCycler. For nucleic acid detection using PCR, the process resembles TaqMan. When a specific DNA sequence is present, a labeled oligonucleotide probe hybridizes between two PCR oligonucleofide primers and is cleaved by the 5′-3′ exonuclease activity of an appropriate polymerase. This frees an eTag reporter from biotin which is part of the probe; the PCR solution is treated with a solution that effectively immobilizes the eTag reporters that have not been cleaved. As in the case of the immunoassays, only free eTag reporters are then electrokinetically injected into the detection system.

The sample 401 is next moved to the avidin section 405. In the avidin section 405 the sample 401 is mixed with Avidin to scavenge eTags that remain bound to PCR probes.

The sample 401 is next moved to the capillary electro-phoresis section 406. Capillary electro-phoresis is common to both immunoassay and nucleic acid assay. The capillary electro-phoresis as used in capillary electro-phoresis section 406 is the same as the capillary electro-phoresis section 305 described above.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A bio-briefcase apparatus for analyzing a sample for the presence of biological agents, comprising:
a housing,
an immunoassay section operatively connected to said housing, or
a nucleic acid assay section operatively connected to said housing.

2. The bio-briefcase apparatus for analyzing a sample for the presence of biological agents of claim 1 wherein said immunoassay section includes an antibody coupling section.

3. The bio-briefcase apparatus for analyzing a sample for the presence of biological agents of claim 1 wherein said immunoassay section includes an antibody coupling section, an antibody reagent section, and microfluidics for moving and mixing the sample.

4. The bio-briefcase apparatus for analyzing a sample for the presence of biological agents of claim 1 wherein said immunoassay section includes a photoactivate section.

5. The bio-briefcase apparatus for analyzing a sample for the presence of biological agents of claim 1 wherein said immunoassay section includes a photoactivate section with exposure of the sample to light.

6. The bio-briefcase apparatus for analyzing a sample for the presence of biological agents of claim 1 wherein said immunoassay section includes a scavenge bound eTags section.

7. The bio-briefcase apparatus for analyzing a sample for the presence of biological agents of claim 1 wherein said immunoassay section includes a scavenge bound eTags section, a reagent section, and microfluidics for moving and mixing the sample to scavenge eTags.

8. The bio-briefcase apparatus for analyzing a sample for the presence of biological agents of claim 1 wherein said immunoassay section includes a capillary electro-phoresis section.

9. The bio-briefcase apparatus for analyzing a sample for the presence of biological agents of claim 1 wherein said nucleic acid assay section includes a lysis section for lysing the sample.

10. The bio-briefcase apparatus for analyzing a sample for the presence of biological agents of claim 1 wherein said nucleic acid assay section includes an acoustics lysis section for lysing the sample.

11. The bio-briefcase apparatus for analyzing a sample for the presence of biological agents of claim 1 wherein said nucleic acid assay section includes a mechanical lysis section for lysing the sample.

12. The bio-briefcase apparatus for analyzing a sample for the presence of biological agents of claim 1 wherein said nucleic acid assay section includes a DNA-capture pillar chip section.

13. The bio-briefcase apparatus for analyzing a sample for the presence of biological agents of claim 1 wherein said nucleic acid assay section includes a DNA-capture pillar chip section that concentrates and filters DNA from the sample.

14. The bio-briefcase apparatus for analyzing a sample for the presence of biological agents of claim 1 wherein said nucleic acid assay section includes a PCR section to perform PCR amplification.

15. The bio-briefcase apparatus for analyzing a sample for the presence of biological agents of claim 1 wherein said nucleic acid assay section includes a flow-through thermal chamber PCR section for performing PCR amplification.

16. The bio-briefcase apparatus for analyzing a sample for the presence of biological agents of claim 1 wherein said nucleic acid assay section includes an avidin section for mixing the sample with Avidin to scavenge eTags.

17. The bio-briefcase apparatus for analyzing a sample for the presence of biological agents of claim 1 wherein said nucleic acid assay section includes a capillary electro-phoresis section.

18. A bio-briefcase apparatus for analyzing a sample for the presence of biological agents, comprising:
a housing,
an immunoassay means operatively connected to said housing for determining the presence of biological agents, and
a nucleic acid assay means operatively connected to said housing for determining the presence of biological agents.

19. The bio-briefcase apparatus for analyzing a sample for the presence of biological agents of claim 18 wherein said immunoassay means includes an antibody coupling section, photoactivate section that exposes the sample to light, an antibody reagent section, a scavenge bound eTags section for moving and mixing the sample to scavenge eTags, a capillary electro-phoresis section, and microfluidics for moving and mixing the sample.

20. The bio-briefcase apparatus for analyzing a sample for the presence of biological agents of claim 18 wherein said nucleic acid assay means includes a lysis section for lysing the sample, a DNA-capture pillar chip section that concentrates and filters DNA from the sample, a PCR section to perform PCR amplification, an avidin section for mixing the sample with Avidin to scavenge eTags, a capillary electro-phoresis section, and microfluidics for moving and mixing the sample.
